(19) United States
(12) Patent Application Publication
(10) Pub. No.: US 2014/0228840 A1
(43) Pub. Date: Aug. 14, 2014

(54) COMPOSITE COMPOSITIONS AND APPLICATIONS THEREOF

(71) Applicant: Wake Forest University Health Sciences, Winston-Salem, NC (US)

(72) Inventors: Joel L. Berry, Birmingham, AL (US); David L. Carroll, Winston-Salem, NC (US); Pierre H. Rolland, Marseilles (FR)

(73) Assignee: Wake Forest University, Winston-Salem, NC (US)

(21) Appl. No.: 14/348,281

(22) PCT Filed: Sep. 28, 2012
(86) PCT No.: PCT/US2012/057965

§ 371 Date: Mar. 28, 2014

Related U.S. Application Data
(60) Provisional application No. 61/540,252, filed on Sep. 28, 2011.

(51) Int. Cl. A61K 47/02; A61K 38/39; A61B 18/14

(52) U.S. Cl. CPC A61K 47/02; A61B 18/14; A61K 38/39
USPC 606/41; 514/17.2; 424/489; 424/94.64; 424/776; 424/520; 514/54

(57) ABSTRACT

In one aspect, composite compositions are described herein. In some embodiments, a composite composition comprises an embolic agent and a plurality of nanoparticles dispersed in the embolic agent, wherein a portion of the nanoparticles are individually dispersed in the embolic agent.

### RELATED APPLICATION DATA

The present invention hereby claims priority under 35 U.S.C. §119(e) to U.S. Provisional Patent Application Ser. No. 61/540,252 filed Sep. 28, 2011, which is hereby incorporated by reference in its entirety.

### FIELD

The present invention relates to composite compositions and methods of using composite compositions for the treatment of diseased tissue.

### BACKGROUND

One of the greatest difficulties in treating cancer is the destruction of healthy tissue in addition to the targeted diseased tissue. For example, in radiofrequency ablation (RFA) therapies, an alternating electric field is applied to a target area in a patient using a probe, such as a needle electrode inserted into the target tissue. Ablation of tissue in the target area is caused by heating during electrode application of the radiofrequency energy. However, RFA can be non-specific, leading to the undesirable killing of healthy tissue within or adjacent to the target area. Moreover, chemotherapy and radiation therapy can also kill many healthy cells in addition to killing cancer cells. This destruction of healthy tissue in addition to unhealthy tissue is a major drawback of many cancer therapies, significantly reducing their beneficial effect for patients in many instances.

### SUMMARY

In one aspect, composite compositions are described herein which can be used in the treatment of diseased tissue, including cancer tissue. Composite compositions described herein, in some embodiments, can reduce or mitigate damage to healthy tissue during the destruction of diseased tissue.

In some embodiments, a composite composition comprises an embolic agent and a plurality of nanoparticles dispersed in the embolic agent, wherein a portion of the nanoparticles are individually dispersed in the embolic agent. The nanoparticles can be dispersed throughout the embolic agent. In some embodiments, for example, the nanoparticles are dispersed throughout the embolic agent uniformly or substantially uniformly. In some embodiments, the nanoparticles are not clustered or are not substantially clustered in the embolic agent. The embolic agent can have a kinematic viscosity permitting intravascular introduction of the composite composition. In some embodiments, a composite composition described herein further comprises at least one radiopaque material.

In another aspect, a composite composition described herein comprises a carrier and at least one cluster of nanoparticles, the carrier comprising an embolic agent and one or more regions of decomposed embolic agent, wherein the nanoparticle cluster is disposed in the embolic agent. In some embodiments, the nanoparticle cluster is disposed in the embolic agent adjacent to one or more regions of the decomposed embolic agent. A composite composition, in some embodiments, comprises a plurality of nanoparticle clusters disposed in the embolic agent adjacent to one or more regions of the decomposed embolic agent. One or more nanoparticle clusters can be fixed in or confined to the embolic agent. Moreover, in some embodiments, nanoparticles of a cluster in the embolic agent are not substantially decomposed or ablated.

In another aspect, biological environments are described herein. In some embodiments, a biological environment comprises diseased tissue and a composite composition disposed in the diseased tissue, the composite composition comprising an embolic agent and a plurality of nanoparticles dispersed in the embolic agent, wherein a portion of the nanoparticles are individually dispersed in the embolic agent. In some embodiments, the nanoparticles are dispersed throughout the embolic agent. The nanoparticles can be dispersed throughout the embolic agent uniformly or substantially uniformly. In some embodiments, the nanoparticles are not clustered or are not substantially clustered in the embolic agent. Further, the nanoparticles, in some embodiments, are fixed in or confined to the embolic agent and are not released from the embolic agent into the diseased tissue or any surrounding healthy tissues.

In another embodiment, a biological environment comprises diseased tissue and a composite composition disposed in the diseased tissue, the composite composition comprising a carrier and at least one cluster of nanoparticles, the carrier comprising an embolic agent and one or more regions of decomposed embolic agent, wherein the nanoparticle cluster is disposed in the embolic agent. In some embodiments, the nanoparticle cluster is disposed in the embolic agent adjacent to one or more regions of the decomposed embolic agent. A composite composition, in some embodiments, comprises a plurality of nanoparticle clusters disposed in the embolic agent adjacent to one or more regions of the decomposed embolic agent. One or more nanoparticle clusters, in some embodiments, are fixed in or confined to the embolic agent and are not released into the diseased tissue or healthy tissue. Moreover nanoparticles of a cluster in the embolic agent are not substantially decomposed or ablated.

In some embodiments of diseased tissue comprising a composite composition described herein, at least a portion of the diseased tissue is ablated or killed within an ablation zone.

In another aspect, methods of treating disease are described herein. In some embodiments, a method of treating disease comprises providing a composite composition comprising an embolic agent and a plurality of nanoparticles dispersed in the embolic agent, disposing the composite composition in a biological environment, and providing thermal energy to the biological environment with the composite composition by exposing the composite composition to radiofrequency energy. The radiofrequency energy, in some embodiments, comprises an alternating electric field. The radiofrequency energy can be supplied by a radiofrequency probe. Further, the biological environment can comprise diseased tissue.

In some embodiments, a method of treating disease described herein further comprises ablating or killing at least a portion of the diseased tissue with the thermal energy to provide an ablation zone. A method of treating disease described herein can further comprise decomposing at least one region of the embolic agent and clustering the nanoparticles in one or more regions of non-decomposed embolic agent. In some embodiments, for example, the nanoparticles are clustered in embolic agent adjacent to one or more regions of decomposed embolic agent. One or more nanoparticle clusters can be fixed in or confined to the embolic agent and are not released from the embolic agent into the diseased tissue or healthy tissue. Moreover, in some embodiments, nanoparticles of a cluster in the embolic agent are not substantially decomposed or ablated.

In another aspect, a method of treating diseased tissue by radiofrequency thermal ablation comprises disposing in the diseased tissue a material having a dielectric loss factor greater than the dielectric loss factor of the diseased tissue to define a predetermined cellular killing zone in the diseased tissue and providing thermal energy to the diseased tissue by exposing the diseased tissue and the material to radiofrequency energy. The predetermined cellular killing zone in the tissue, in some embodiments, is restricted to the region of material having the greater dielectric loss factor. In some embodiments, the predetermined cellular killing zone includes the region of greater dielectric loss factor material and an adjacent region in the tissue having dimension(s) up to about 50% the diameter of the region of greater dielectric loss factor material. The adjacent region, in some embodiments, has dimension(s) up to about 40 percent or up to about 20 percent the diameter of the region of greater dielectric loss factor material. The thermal energy supplied to the diseased tissue by application of radiofrequency energy, in some embodiments, is sufficient to ablate or otherwise kill cells in the predetermined cellular killing zone. In some embodiments, cells outside the predetermined cellular killing zone are not killed or damaged, thereby reducing or precluding damage to healthy tissue during RF ablation procedures. As described herein, the radiofrequency energy can be supplied by a radiofrequency probe.

In another aspect, methods of reducing damage to non-diseased tissue during the treatment of diseased tissue are described herein. In some embodiments, a method comprises reducing damage to non-diseased tissue adjacent to diseased tissue during radiofrequency thermal ablation of the diseased tissue in an ablation zone by restricting formation of an apoptotic cellular region in the non-diseased tissue resulting from the radiofrequency thermal ablation, wherein restricting comprises disposing in the diseased tissue, prior to application of radiofrequency energy, a material having a dielectric loss factor greater than the dielectric loss factor of the diseased tissue. In some embodiments, the material having a greater dielectric loss factor is present in the diseased tissue in an amount sufficient to heat at least a portion of the diseased tissue when exposed to radiofrequency energy. In some embodiments, for example, the material is present in an amount sufficient to thermally ablate at least a portion of the diseased tissue when exposed to radiofrequency energy.

In some embodiments of methods described herein, a material having a dielectric loss factor greater than the diseased tissue comprises a composite comprising an embolic agent and a plurality of nanoparticles dispersed in the embolic agent. In some embodiments, the material of greater dielectric loss factor comprises any composite composition described herein.

In another aspect, treatment systems for diseased tissue are provided. In some embodiments, a treatment system for diseased tissue comprises a source of radiofrequency energy and a thermal induction agent operable for positioning in the diseased tissue, the thermal induction agent comprising a material having a dielectric loss factor greater than the dielectric loss factor of the diseased tissue. The material of the thermal induction agent, in some embodiments, comprises an embolic agent and a plurality of nanoparticles dispersed in the embolic agent. In some embodiments, the material further comprises at least one radiopaque material. In some embodiments, the nanoparticles are dispersed throughout the embolic agent, wherein at least a portion of the nanoparticles are individually dispersed in the embolic agent. In some embodiments, for example, the nanoparticles are not clustered or are not substantially clustered in the embolic agent. The embolic agent can have a kinematic viscosity permitting intravascular introduction of the composite composition.

In some embodiments, the source of radiofrequency energy is a radiofrequency probe. A radiofrequency probe, in some embodiments, has a structure suitable for insertion into a patient. In one embodiment, for example, a radiofrequency probe has a needle-like or tubular structure for insertion into a patient. Alternatively, in some embodiments, a radiofrequency probe has a structure that is not intended to be inserted into a patient. In such embodiments, the radiofrequency probe provides radiofrequency energy from outside the patient.

These and other embodiments are described in greater detail in the detailed description which follows.

### BRIEF DESCRIPTION OF THE DRAWINGS

FIG. 1 is a transmission electron microscopy (TEM) image of multi-walled carbon nanotubes for use composite compositions according to some embodiments described herein.

FIG. 2 is a photograph of an apparatus for preparing composite compositions according to some embodiments described herein.

FIG. 3 is a TEM image of individualized multi-walled carbon nanotubes dispersed in an embolic agent according to some embodiments described herein.

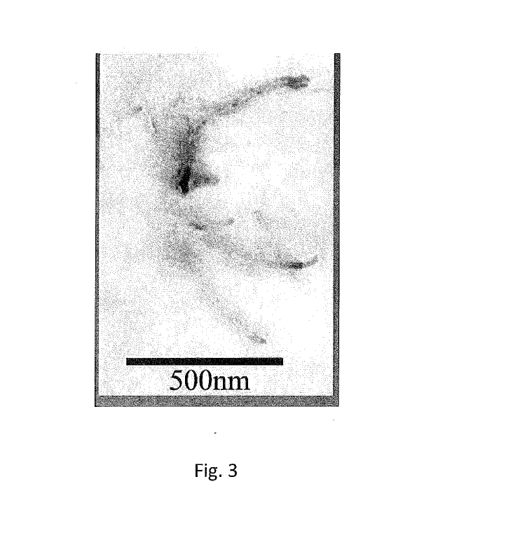

FIG. 4 is an SEM image of multi-walled carbon nanotubes dispersed in an embolic agent according to some embodiments described herein.

FIG. 5 is a TEM image of a composite composition according to one embodiment described herein following exposure to radiofrequency energy.

FIG. 6 illustrates several results of RF ablation (RFA) studies according to some embodiments of methods described herein.

FIG. 7 illustrates several results of RFA studies according to some embodiments of methods described herein.

FIG. 8 illustrates several results of RFA studies according to some embodiments of methods described herein.

FIG. 9 illustrates several results of RFA studies according to some embodiments of methods described herein.

FIG. 10 illustrates several results of RFA studies according to some embodiments of methods described herein.

FIG. 11 illustrates restricting the formation of an apoptotic cellular region in non-diseased tissue resulting from the RF ablation of adjacent diseased tissue according to one embodiment described herein.

### DETAILED DESCRIPTION

Embodiments described herein can be understood more readily by reference to the following detailed description, examples, and drawings. Elements, apparatus, and methods described herein, however, are not limited to the specific embodiments presented in the detailed description, examples, and drawings. It should be recognized that these embodiments are merely illustrative of the principles of the present invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

### I. Composite Compositions

In one aspect, composite compositions are described herein. In some embodiments, a composite composition comprises an embolic agent and a plurality of nanoparticles dispersed in the embolic agent, wherein a portion of the nanoparticles are individually dispersed in the embolic agent. In some embodiments, the nanoparticles are dispersed throughout the embolic agent. In some embodiments, the nanoparticles are dispersed throughout the embolic agent uniformly or substantially uniformly. In some embodiments, the nanoparticles are not clustered or are not substantially clustered in the embolic agent. Moreover, in some embodiments, the nanoparticles have substantial freedom of movement within the embolic agent. Freedom of movement, in some embodiments, refers to the ability of a nanoparticle to shift orientation in response to an applied electric field, such as that associated with radiofrequency energy.

In some embodiments, composite compositions described herein are operable to transfer thermal energy to their surroundings when irradiated with or exposed to radiofrequency energy. For example, in some embodiments, composite compositions described herein are operable to transfer thermal energy to surrounding biological tissue. In some embodiments, composite compositions described herein are operable to heat their surroundings without releasing one or more components of the composite composition to the surroundings. For example, in some embodiments, composite compositions described herein are operable to heat their surroundings without releasing nanoparticles or nanoparticle clusters to the surroundings. In some embodiments, the nanoparticles and/or nanoparticle clusters remain fixed or trapped in the embolic agent of the composite composition.

Nanoparticles of a composite composition described herein can be present in the embolic agent in any amount not inconsistent with the objectives of the present invention. In some embodiments, nanoparticles are present in the embolic agent in an amount ranging from about 0.1 μg/ml to about 5 mg/ml. In some embodiments, nanoparticles are present in the embolic agent in an amount ranging from about 1 μg/ml to about 1 mg/ml or from about 10 μg/ml to about 1 mg/ml. In some embodiments, nanoparticles are present in the embolic agent in an amount ranging from about 50 μg/ml to about 0.5 mg/ml or from about 0.1 mg/ml to about 0.5 mg/ml. In some embodiments, nanoparticles are present in the embolic agent in an amount less than about 0.1 μg/ml or greater than about 5 mg/ml. In some embodiments, nanoparticles are present in the embolic agent in an amount greater than about 10 mg/ml. Moreover, in some embodiments, the concentration of nanoparticles in the embolic agent can be varied according to one or more considerations including, but not limited to, the identity of the environment in which the composite composition is to be disposed, the identity of the nanoparticles, the identity of the embolic agent and/or the desired amount of heat to be provided to the surrounding environment.

Turning now to components of a composite composition described herein, a composite composition described herein comprises an embolic agent. Any embolic agent not inconsistent with the objectives of the present invention can be used in a composite composition described herein.

In some embodiments of composite compositions described herein, the embolic agent has a kinematic viscosity permitting intravascular introduction of the composite composition into tissue. For example, in some embodiments, the embolic agent has a kinematic viscosity permitting intravascular introduction of the composite composition through a microcatheter. A microcatheter, in some embodiments, can have an inner diameter ranging from about 100 μm to about 1500 μm, from about 200 μm to about 1200 μm, or from about 500 μm to about 800 μm.

In some embodiments of composite compositions described herein, the embolic agent has a kinematic viscosity permitting intravascular introduction of the composite composition through a catheter having an inner diameter up to about 3 mm. In some embodiments, the kinematic viscosity of the composite composition can be varied according to one or more considerations including, but not limited to, the amount of the composite composition to be delivered, the identity of the tissue being treated and/or the volume of the tissue to be treated

In some embodiments, the embolic agent is liquid or fluid. In some embodiments, the embolic agent is semi-solid. The embolic agent, in some embodiments, is a non-adhesive embolic agent. In some embodiments, the embolic agent is an adhesive embolic agent.

In some embodiments, the embolic agent comprises polymeric material. In some embodiments, the embolic agent comprises crosslinked polymeric material. In some embodiments, the embolic agent comprises a gel or gelatin. In some embodiments, the embolic agent comprises a hydrogel. In some embodiments, the embolic agent comprises a foam such as a gel foam. In some embodiments, the embolic agent comprises an emulsion. In some embodiments, the embolic agent comprises a lipid emulsion. In some embodiments, the embolic agent comprises self-assembling embolic material.

The embolic agent, in some embodiments, comprises collagen, thrombin, lipiodol, gelatin, acrylic gelatin, tris-acryl gelatin, alginic acid, alginate, cellulose acetate, poly(vinyl acetate), poly(ethylene vinyl alcohol), ethylene vinyl alcohol copolymer (EVOH), a biodegradable poly(hydroxyl acid) or poly(vinyl alcohol) (PVA) or various mixtures thereof. In some embodiments, the embolic agent comprises Marsembol. In some embodiments, the embolic agent comprises an alkyl cyanoacrylate, such as n-butyl cyanoacrylate.

In some embodiments, the embolic agent comprises a particulate material, including microspheres and/or non-spherical particles. Particulate material of an embolic agent can have any size not inconsistent with the objectives of the present invention. In some embodiments, the particulate material comprises deformable or compressible particles. In some embodiments, the particulate material has a particle size and/or compressibility that permits the embolic agent to be delivered through a microcatheter.

As described herein, a composite composition comprises a plurality of nanoparticles dispersed in the embolic agent. In some embodiments, at least a portion of the nanoparticles are individually dispersed in the embolic agent. In some embodiments, surfaces of nanoparticles dispersed in the embolic agent are functionalized to assist in the dispersion. In some embodiments, surfaces of the nanoparticles are not functionalized.

In some embodiments, nanoparticles of composite compositions described herein have an aspect ratio greater than 1. In some embodiments, the nanoparticles have an aspect ratio ranging from about 1.1 to about 10,000. In some embodiments, the nanoparticles have an aspect ratio ranging from about 10 about 1,000 or from about 10 to about 100. The nanoparticles, in some embodiments, have an aspect ratio ranging from about 5 to about 50 or from about 15 to about 40.

In some embodiments, nanoparticles of a composite composition described herein have a length ranging from about 100 nm to about 3 μm or from about 400 nm to about 2 μm. In some embodiments, the nanoparticles have a length ranging from about 500 nm to about 1.5 μm or from about 700 nm to about 1 μm. Nanoparticles, in some embodiments, have a length ranging from about 800 nm to about 1 μm or from about 850 nm to about 950 nm. In some embodiments, the nanoparticles have a length greater than about 1 μm or a length ranging from about 1 μm to about 2 μm. In some embodiments, the nanoparticles have a length ranging from about 1.5 μm to about 2 μm or from about 2 μm to about 2.5 μm. In some embodiments, the nanoparticles have a length ranging from about 2.5 μm to about 3 μm or have a length greater than about 3 μm. In some embodiments, the nanoparticles have a length ranging from about 1 μm to about 20 μm.

In some embodiments, the nanoparticles have a diameter less than about 200 nm. In some embodiments, the nanoparticles have a diameter ranging from about 5 nm to about 150 nm or from about 10 nm to about 100 mm In some embodiments, the nanoparticles have a diameter ranging from about 20 nm to about 80 nm. In some embodiments, the nanoparticles have a diameter ranging from about 30 nm to about 50 nm.

Nanoparticles of composite compositions described herein can comprise any type of nanoparticle not inconsistent with the objectives of the present invention. In some embodiments, for example, the nanoparticles comprise carbon nanoparticles. In some embodiments, carbon nanoparticles comprise carbon nanotubes, including single-walled carbon nanotubes (SWNT) and multi-walled carbon nanotubes (MWNT). Carbon nanotubes, in some embodiments, have branched structures. Branched structures, in some embodiments, comprise multiple branches, Y branches, Y branches with multiple branches and multi-level Y branches.

Carbon nanotubes, in some embodiments, can be doped with boron, nitrogen or combinations thereof. In some embodiments, for example, doped carbon nanotubes comprise boron in an amount ranging from about 0.01 weight percent to about 10 weight percent. In some embodiments, doped carbon nanotubes comprise about 5 weight percent boron. In some embodiments, doped carbon nanotubes comprise nitrogen in an amount ranging from about 0.01 weight percent to about weight 30 percent or from about 5 weight percent to about 25 weight percent. In some embodiments, doped carbon nanotubes comprise nitrogen in an amount greater than about 30 weight percent. In some embodiments, doped carbon nanotubes comprise from about 10 weight percent to about 20 weight percent nitrogen. In some embodiments, doped carbon nanotubes comprise less than about 1 weight percent nitrogen.

In some embodiments, carbon nanotubes comprise transition metals, including iron, cobalt, nickel, silver or combinations thereof. In some embodiments, a carbon nanotube comprises at least about 0.01 weight percent of a transition metal. In some embodiments, a carbon nanotube comprises a transition metal in an amount ranging from about 0.5 weight percent to about 3 weight percent or from about 1 weight percent to about 2 weight percent. In some embodiments, a transition metal is disposed in the cavity of a nanotube or between walls of a MWNT. In some embodiments, a transition metal is attached to a surface of a nanotube or incorporated into the lattice of the nanotube. Moreover, in some embodiments, a carbon nanotube has anti-microbial properties. In some embodiments, for example, a carbon nanotube comprising silver has antibacterial properties.

In some embodiments, a carbon nanotube comprises at least one positive magnetic resonance (T1) contrast agent. In some embodiments, a positive contrast agent includes chemical species comprising gadolinium, such as gadolinium chloride. In some embodiments, the at least one magnetic resonance contrast agent is disposed within the nanotube. In some embodiments, the at least one magnetic resonance contrast agent is disposed on a surface of the carbon nanotube. In some embodiments, carbon nanotubes comprising iron and/or a positive contrast agent are doped with nitrogen and/or boron.

In some embodiments, the nanoparticles comprise graphene, nanohorns, fullerite or mixtures thereof.

Moreover, in some embodiments, nanoparticles of a composite composition described herein comprise an inorganic nanoparticle. In some embodiments, inorganic nanoparticles comprise nanoshells, nanorods, nanowires, nanotubes, or mixtures thereof. Inorganic nanoparticles, in some embodiments, comprise metals, including transition metals, noble metals, alkali metals and alkaline-earth metals. Inorganic nanoparticles, in some embodiments, comprise metal oxides such as tungsten oxide, vanadium oxide, titanium oxide or combinations thereof In some embodiments, inorganic nanoparticles comprise boron nitride. In some embodiments, inorganic nanoparticles comprise semiconductor materials, including II/VI and III/V semiconductors. In some embodiments, an inorganic nanoparticle comprises a nanotube or nanorod. In some embodiments, an inorganic nanotube or nanorod comprises one or more of tungsten oxide, vanadium oxide, titanium oxide or boron nitride.

In another aspect, a composite composition described herein comprises a carrier and at least one cluster of nanoparticles, the carrier comprising an embolic agent and one or more regions of decomposed embolic agent, wherein the nanoparticle cluster is disposed in the embolic agent. In some embodiments, the nanoparticle cluster is disposed in the embolic agent adjacent to one or more regions of the decomposed embolic agent. A composite composition, in some embodiments, comprises a plurality of nanoparticle clusters disposed in the embolic agent adjacent to one or more regions of the decomposed embolic agent. In some embodiments, all or substantially all of the nanoparticles of the composite are part of one or more clusters in the embolic agent. As described further herein, one or more regions of decomposed embolic agent can be produced by exposure of the composite composition to radiofrequency energy.

In some embodiments, the one or more regions of decomposed embolic agent comprise thermally decomposed embolic agent. In some embodiments, the one or more regions of decomposed embolic agent comprise chemically decomposed embolic agent. In some embodiments, the one or more regions of decomposed embolic agent comprise physically decomposed embolic agent. In some embodiments, the one or more regions of decomposed embolic agent comprise phase segregated embolic agent.

In some embodiments, one or more nanoparticle clusters are fixed in or confined to the embolic agent. Moreover, in some embodiments, nanoparticles of a cluster in the embolic agent are not substantially decomposed or ablated.

### II. Biological Environments

In another aspect, biological environments are described herein. In some embodiments, a biological environment comprises a diseased tissue and a composite composition disposed in the diseased tissue, the composite composition comprising an embolic agent and a plurality of nanoparticles dispersed in the embolic agent, wherein a portion of the nanoparticles are individually dispersed in the embolic agent. In some embodiments, the nanoparticles are dispersed throughout the embolic agent. In some embodiments, the nanoparticles are dispersed throughout the embolic agent uniformly or substantially uniformly. In some embodiments, the nanoparticles are not clustered or are not substantially clustered in the embolic agent. In some embodiments, the nanoparticles are fixed in or confined to the embolic agent and are not released from the embolic agent into the diseased tissue or any surrounding healthy tissues.

In another embodiment, a biological environment comprises a diseased tissue and a composite composition disposed in the diseased tissue, the composite composition comprising a carrier and at least one cluster of nanoparticles, the carrier comprising an embolic agent and one or more regions of decomposed embolic agent, wherein the nanoparticle cluster is disposed in the embolic agent. In some embodiments, the nanoparticle cluster is disposed in the embolic agent adjacent to one or more regions of the decomposed embolic agent. A composite composition, in some embodiments, comprises a plurality of nanoparticle clusters disposed in the embolic agent adjacent to one or more regions of the decomposed embolic agent. In some embodiments, one or more nanoparticle clusters are fixed in or confined to the embolic agent and are not released into the diseased tissue or healthy tissue.

In some embodiments, the one or more regions of decomposed embolic agent comprise thermally decomposed embolic agent. In some embodiments, the one or more regions of decomposed embolic agent comprise chemically decomposed embolic agent. In some embodiments, the one or more regions of decomposed embolic agent comprise physically decomposed embolic agent. In some embodiments, the one or more regions of decomposed embolic agent comprise phase segregated embolic agent.

In some embodiments of a biological environment, any embolic agent described in Section I hereinabove can be used for the embolic agent of the composite composition. Moreover, in some embodiments, any of the nanoparticles described in Section I hereinabove can be used as the nanoparticle component of the composite composition.

In some embodiments, the diseased tissue of a biological environment comprises tumor tissue. In some embodiments, the tumor tissue comprises a fibroid. In some embodiments, the tumor tissue comprises a myoma. In some embodiments, the diseased tissue comprises cancerous tissue. In some embodiments, the diseased tissue comprises liver, lung, or bone cancer. In some embodiments, the diseased tissue comprises kidney cancer. In some embodiments, composite compositions are disposed in the vasculature of cancer tissue. In some embodiments, cancerous tissue treated with composite compositions described herein is malignant. In some embodiments, cancerous tissue treated with composite compositions described herein is benign.

In some embodiments of a biological environment, at least a portion of the diseased tissue is ablated within an ablation zone. In some embodiments, substantially all of the diseased tissue is ablated within an ablation zone. An ablation zone, in some embodiments, comprises a spatial region wherein all or substantially all of the tissue is ablated. In some embodiments, the ablation zone comprises a thermal ablation zone.

Composite compositions described herein, in some embodiments, permit ablation of diseased tissue with reduced negative impact on surrounding healthy tissue. In some embodiments of biological environments described herein wherein at least a portion of the diseased tissue is ablated within an ablation zone, the biological environment further comprises a non-diseased tissue adjacent to the diseased tissue. In some embodiments, the non-diseased tissue comprises an apoptotic cellular region adjacent to the ablation zone, the apoptotic region extending into the non-diseased tissue a distance less than about 50 percent of the diameter of the ablation zone. In some embodiments, the apoptotic cellular region extends into the non-diseased tissue a distance less than about 40 percent or less than about 30 percent of the diameter of the ablation zone. In some embodiments, the apoptotic cellular region extends into the non-diseased tissue a distance less than about 20 percent or less than about 15 percent of the diameter of the ablation zone. In some embodiments, the apoptotic cellular region is formed or induced as a result of the ablation of the diseased tissue.

In some embodiments, less than about 50 percent of non-diseased cells in the apoptotic region display apoptotic behavior. In some embodiments, less than about 40 percent or less than about 30 percent of non-diseased cells in the apoptotic region display apoptotic behavior. In some embodiments, less than about 20 percent or less than about 10 percent of non-diseased cells in the apoptotic region display apoptotic behavior.

### III. Methods of Treating Disease

In another aspect, methods of treating disease are described herein. In some embodiments, a method of treating disease comprises providing a composite comprising an embolic agent and a plurality of nanoparticles dispersed in the embolic agent, disposing the composite composition in a biological environment, and providing thermal energy to the biological environment with the composite composition by exposing the composite composition to radiofrequency energy. The composite composition can comprise any composite composition described in Section I hereinabove.

In some embodiments, the biological environment comprises diseased tissue. In some embodiments, the diseased tissue comprises tumor tissue. In some embodiments, the tumor tissue comprises a fibroid. In some embodiments, the tumor tissue comprises a myoma. In some embodiments, the diseased tissue comprises cancerous tissue. In some embodiments, for example, the diseased tissue comprises liver, lung, or bone cancer. In some embodiments, the diseased tissue comprises kidney cancer.

In some embodiments of methods described herein, the composite composition is disposed in the vasculature of the diseased tissue. In some embodiments, the composite composition is disposed in the vasculature of cancerous tissue. In being disposed in the vasculature of the diseased tissue, the composite composition, in some embodiments, does not enter diseased cells or become associated with receptors of cellular membranes of diseased cells.

Methods described herein, in some embodiments, permit the ablation or substantial ablation of diseased tissue using RFA with reduced radiofrequency (RF) exposure times. In some embodiments, reduced RF exposure times are demonstrated by reductions in time to roll-off, as further illustrated in Example 4 herein. In some embodiments of methods described herein, time to roll-off is reduced by at least 5 percent in comparison to an RF treatment method wherein the composite composition is not disposed in the diseased tissue. In some embodiments, time or roll-off is reduced by at least about 10 percent or by at least 20 percent. Time to roll-off, in some embodiments, is reduced by at least about 30 percent or at least about 40 percent. In some embodiments, time to roll-off is reduced by about 5 percent to about 50 percent. In some embodiments, reductions in RF exposure times can limit damage to healthy or non-diseased tissue in the vicinity of the diseased tissue.

In some embodiments wherein the biological environment comprises diseased tissue, a method of treating disease described herein further comprises ablating at least a portion of the diseased tissue to provide an ablation zone. An ablation zone, in some embodiments, comprises a spatial region wherein all or substantially all of the tissue is ablated. In some embodiments, the ablation zone comprises a thermal ablation zone. In some embodiments of methods described herein, thermal energy provided by the composite composition ablates or assists in ablating the diseased tissue.

In some embodiments, the biological environment further comprises non-diseased tissue. In some embodiments, the non-diseased tissue comprises an apoptotic cellular region adjacent to the ablation zone, the apoptotic cellular region extending into the non-diseased tissue a distance up to about 50 percent of the diameter of the ablation zone. In some embodiments, the apoptotic cellular region extends into the non-diseased tissue a distance up to about 40 percent, up to about 20 percent or up to about 15 percent of the diameter of the ablation zone. In some embodiments, the apoptotic cellular region is formed or induced as a result of the ablation of the diseased tissue.

In some embodiments, less than about 50 percent of non-diseased cells in the apoptotic region display apoptotic behavior. In some embodiments, less than about 40 percent, less than about 30 percent, less than about 20 percent, or less than about 10 percent of non-diseased cells in the apoptotic region display apoptotic behavior.

In some embodiments, a method of treating disease described herein further comprises decomposing one or more regions of the embolic agent of the composite composition and clustering the nanoparticles in one or more regions of non-decomposed embolic agent. In some embodiments, the nanoparticles are clustered in non-decomposed embolic agent adjacent to a region of decomposed embolic agent. In some embodiments, the clusters of nanoparticles are confined to or fixed in one or more regions of the non-decomposed embolic agent. In being confined to or fixed in non-decomposed embolic agent, the clusters of nanoparticles are not released from the embolic agent into the diseased tissue or any surrounding healthy tissues.

In some embodiments, the one or more regions of decomposed embolic agent comprise thermally decomposed embolic agent. In some embodiments, the one or more regions of decomposed embolic agent comprise chemically decomposed embolic agent. In some embodiments, the one or more regions of decomposed embolic agent comprise physically decomposed embolic agent. In some embodiments, the one or more regions of decomposed embolic agent comprise phase segregated embolic agent. In some embodiments, for example, one or more regions of the embolic agent are thermally decomposed during exposure of the composite composition to radiofrequency energy.

In another aspect, a method of treating diseased tissue by radiofrequency thermal ablation comprises disposing in the diseased tissue a material having a dielectric loss factor greater than the dielectric loss factor of the diseased tissue to define a predetermined cellular killing zone in the diseased tissue and providing thermal energy to the diseased tissue by exposing the diseased tissue and the material to radiofrequency energy. The predetermined cellular killing zone in the tissue, in some embodiments, is restricted to the region of material having the greater dielectric loss factor. In some embodiments, the predetermined cellular killing zone includes the region of greater dielectric loss factor material and an adjacent region having dimension(s) up to about 50% the diameter of the region of greater dielectric loss factor material. The adjacent region, in some embodiments, has dimension(s) up to about 40 percent or up to about 20 percent the diameter of the region of greater dielectric loss factor material. The thermal energy supplied to the diseased tissue by application of radiofrequency energy, in some embodiments, is sufficient to ablate or otherwise kill cells in the predetermined cellular killing zone. In some embodiments, cells outside the predetermined cellular killing zone are not killed or damaged, thereby reducing or precluding damage to healthy tissue during RF ablation procedures. As described herein, the radiofrequency energy can be supplied by a radiofrequency probe. In some embodiments, the diseased tissue in the predetermined cellular killing zone is cancerous tissue.

In another aspect, methods of reducing damage to non-diseased tissue during the treatment of diseased tissue are described herein. In some embodiments, a method comprises reducing damage to non-diseased tissue adjacent to diseased tissue during radiofrequency thermal ablation of the diseased tissue in an ablation zone by restricting formation of an apoptotic cellular region in the non-diseased tissue resulting from the radiofrequency thermal ablation, wherein restricting comprises disposing in the diseased tissue, prior to application of radiofrequency energy, a material having a dielectric loss factor greater than the dielectric loss factor of the diseased tissue. In some embodiments, the material having a greater dielectric loss factor is present in the diseased tissue in an amount sufficient to heat at least a portion of the diseased tissue when exposed to radiofrequency energy. In some embodiments, for example, the material is present in an amount sufficient to thermally ablate at least a portion of the diseased tissue when exposed to radiofrequency energy.

In some embodiments, a material having a dielectric loss factor greater than the diseased tissue comprises a composite described herein comprising an embolic agent and a plurality of nanoparticles dispersed in the embolic agent. In some embodiments, the material comprises any composite composition described in Section I hereinabove. Moreover, in some embodiments, a material having a dielectric loss factor greater than the diseased tissue can comprise any material not inconsistent with the objectives of the present invention. In some embodiments, for example, a suitable material has a dielectric loss factor enabling the material to provide sufficient thermal energy to ablate or assist in ablating diseased tissue within an ablation zone when exposed to radiofrequency energy.

In some embodiments, materials having a dielectric loss factor greater than the diseased tissue comprise polymeric materials or polymeric composites. In some embodiments, materials having a dielectric loss factor greater than the diseased tissue comprise one or more gels, including hydrogels. In some embodiments, a material having a dielectric loss factor greater than the diseased tissue comprises one or more ionic or dipole components. In some embodiments, for example, polymeric species of a material can be charged. In some embodiments, a material having a dielectric loss factor greater than the diseased tissue can comprise one or more salts. In some embodiments of methods of treating diseased tissue and methods of reducing damage to non-diseased tissue during the RF ablation, the dielectric loss factor of the diseased tissue can be estimated according to methods and techniques disclosed in S. Seker and H. Abatay, “New frequency-dependent parametric modeling of dielectric materials,” Int. J. Electron. Commun. (AEU) 60 (2006), 320-327, the entirety of which is incorporated herein by reference. In some embodiments, for example, equations (2) and (3) in Seker et al. can be used in estimating the dielectric loss factor of diseased tissue.

In some embodiments, the material disposed in the diseased tissue prior to application of radiofrequency energy has dielectric loss factor at least about 10 percent greater than the dielectric loss factor of the diseased tissue. In some embodiments, the material has a dielectric loss factor at least about 30 percent or at least about 50 percent greater than the dielectric loss factor of the diseased tissue. In some embodiments, the material has a dielectric loss factor at least about 70 percent or at least about 100 percent greater than the dielectric loss factor of the diseased tissue. In some embodiments, the material has a dielectric loss factor ranging from about 10 percent to about 1000 percent greater than the dielectric loss factor of the diseased tissue.

In some embodiments of methods treating diseased tissue and methods of reducing damage to non-diseased tissue during the RF ablation, the diseased tissue comprises tumor tissue. In some embodiments, the tumor tissue comprises a fibroid. In some embodiments, the tumor tissue comprises a myoma. In some embodiments, the diseased tissue comprises cancerous tissue. The cancerous tissue can comprise any cancerous tissue not inconsistent with the objectives of the present invention. In some embodiments, the diseased tissue comprises liver, liver, or bone cancer. In some embodiments, the diseased tissue comprises kidney cancer.

In some embodiments, an ablation zone of a method of reducing damage to non-diseased tissue comprises a spatial region wherein all or substantially all of the tissue is ablated. In some embodiments, the formation of the apoptotic cellular region is restricted to a region adjacent to the ablation zone and extending into the non-diseased tissue a distance up to about 50 percent of the diameter of the ablation zone. In some embodiments, the apoptotic cellular region is restricted to a region adjacent to the ablation zone and extending into the non-diseased tissue a distance up to about 40 percent or up to about 20 percent the diameter of the ablation zone. In some embodiments, the apoptotic cellular region extends into the non-diseased tissue a distance up to about 15 percent of the diameter of the ablation zone.

Moreover, in some embodiments, less than about 40 percent of cells in the apoptotic region display apoptotic behavior. In some embodiments, less than about 30 percent, less than about 20 percent or less than about 10 percent of cells in the apoptotic region display apoptotic behavior.

### IV. Treatment Systems for Diseased Tissue

In another aspect, treatment systems for diseased tissue are provided. In some embodiments, a treatment system for diseased tissue comprises a source of radiofrequency energy and a thermal induction agent operable for positioning in the diseased tissue, the thermal induction agent comprising a material having a dielectric loss factor greater than the dielectric loss factor of the diseased tissue. The material of the thermal induction agent, in some embodiments, comprises an embolic agent and a plurality of nanoparticles dispersed in the embolic agent. In some embodiments, the material further comprises at least one radiopaque material. A thermal induction agent refers to a species operable to transfer heat energy to its surrounding environment when exposed to radiofrequency energy.

In some embodiments of treatment systems described herein, at least a portion of the nanoparticles are individually dispersed in the embolic agent. In some embodiments, the nanoparticles are dispersed throughout the embolic agent uniformly or substantially uniformly. The nanoparticles, in some embodiments, are not clustered or are not substantially clustered in the embolic agent. Moreover, in some embodiments, the nanoparticles have freedom of movement within the embolic agent. Freedom of movement, in some embodiments, refers to the ability of a nanoparticle to shift orientation in response to an applied electric field, such as that associated with radiofrequency energy.

In some embodiments, the material of the thermal induction agent can have any construction described in Section I hereinabove for a composite composition. In some embodiments, the source of radiofrequency energy is a radiofrequency probe.

Some embodiments described herein are further illustrated in the following non-limiting examples.

### EXAMPLE 1

### Multi-walled Carbon Nanotubes (MWNTs)

Multi-walled carbon nanotubes (MWNT) were produced by chemical vapor deposition (CVD). The synthesis of the MWNT was executed in a two-stage quartz furnace (diameter ˜45 mm, work length ˜45 mm). Hydrogen was used as the carrier gas with a flow rate of about 320 sscm, and the preheater of the two-stage furnace was maintained at 160° C. About 2.7 percent of ferrocine by weight was dissolved in toluene, and the resulting solution was injected into the preheater at a rate of 5 ml/hr. The temperature of the furnace ranged from 600° C. to 900° C., and the growth time was set to one hour. The resulting MWNT were 50 nm in diameter and cut by sonication to about 900 nm in length.

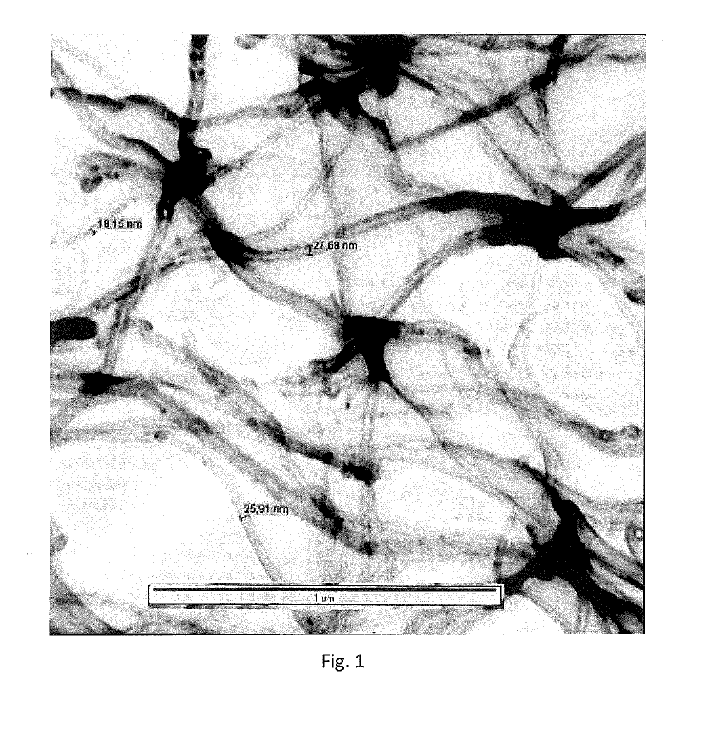

Three (3) mg of MWNT were vortexed in 0.1 ml of distilled water and sonicated 5 times, each sonication time lasting 15 seconds with a 10 second vortexing between sonications, to provide an aqueous suspension of the MWNT. FIG. 1 illustrates a TEM image of the aqueous MWNT suspension according to one embodiment.

### EXAMPLE 2

### Preparation of Embolic Agent—Marsembol

Embolic agent Marsembol was prepared as follows. First, a gelatin-resorcinol mixture was prepared by adding 37.5 g Rousselot 250 bloom PS gelatin and 2.5 g resorcinol to 48.75 ml distilled water containing 1.25 g calcium chloride. The mixture was placed in a water bath at 40° C. and gently stirred for at least one hour to give a clear, honey-like, dense and viscous mixture.

Next, a desired volume of the gelatin-resorcinol mixture was mixed with lipiodol (ethyl esters of iodized fatty acids of poppy seed oil, Guerbet, Roissy, France) in a ratio of 0.5 to 2 (by volume) in a thermostated water bath at 40° C. for 15 minutes or more, giving a cross-linked gelatin loaded with lipiodol. The gelatin was further cross-linked by mixing the gelatin mixture with an aqueous solution containing 0.9% glutaraldehyde and 15.5% formaldehyde in a ratio of 2.5 to 0.1 (by volume) at 40° C. for one hour.

Finally, excess or unreacted aldehyde in the mixture was neutralized by adding 100 mM of aqueous glycine (free or in chlorhydrate form) in a ratio of 1 to 5 (by volume). Additional detail regarding the preparation and characterization of Marsembol can be found in Vidal et al., “Effectiveness of endovascular embolization with a collagen-based embolic agent (Marsembol) in an animal model,” J. Vasc. Interv. Radiol. (September 2010), 21 (9), 1419-1423, which is hereby incorporated by reference in its entirety.

### EXAMPLE 3

### MWNTs Dispersed in Marsembol

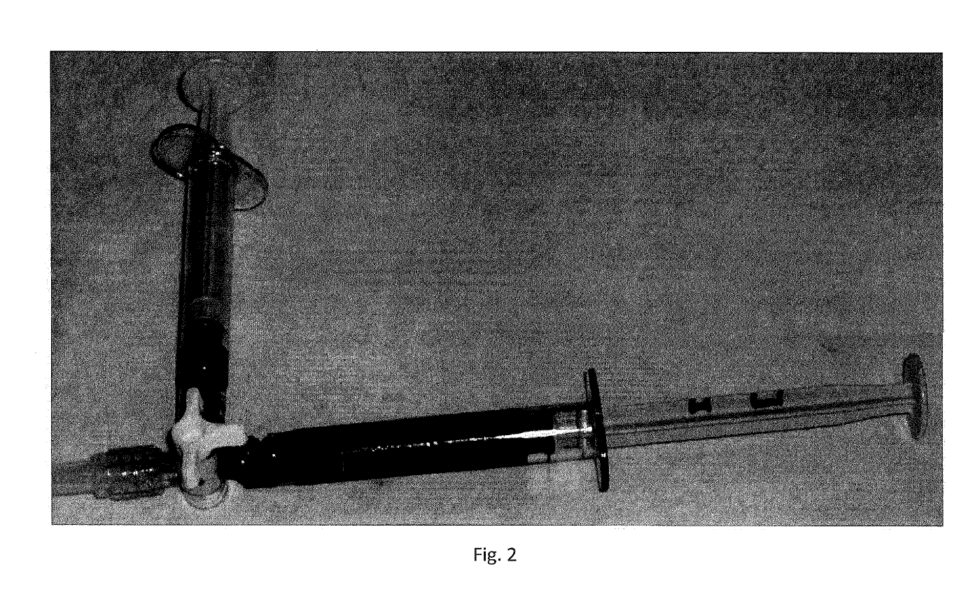

100 μl of the MWNT aqueous suspension of Example 1 was mixed with 2.5 ml of the embolic agent Marsembol of Example 2 as follows to produce a composite composition according to some embodiments described herein. The 100 μl MWNT sample were mixed with 2.5 ml Marsembol using two 5-ml syringes linked with a three-way stopcock, as illustrated in FIG. 2. The MWNT aqueous suspension was loaded into the first syringe, and the Marsembol was loaded into the second syringe. The MWNT aqueous suspension and Marsembol were subsequently mixed by transferring the compositions between the syringes at least 10 times.

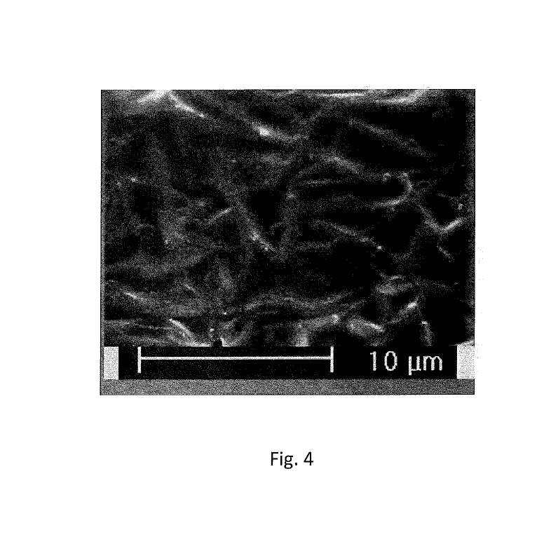

As illustrated in FIG. 4, at least a portion of the MWNT were individually dispersed in the Marsembol embolic agent. This is in contrast to the MWNT aqueous dispersion of FIG. 2 where the MWNT are clustered. Moreover, as illustrated in FIG. 4, the MWNT demonstrated intimate contact with the Marsembol embolic agent.

### EXAMPLE 4

### Radiofrequency Ablation

A series of radiofrequency ablation studies in animal models were conducted as follows. All animals were handled in accordance with the Guide for the Care and Use of Laboratory Animals from the U.S. National Research Council. In addition, the regional Care and Use Committee for experimental research approved the experimental protocol used. Three female baboons (Papio Anubis, 5-7 years old, 15-17 kg in weight, obtained from Station de primatologie CNRS, D56 Rousset 13790 France) and six pigs (8 months old, 65±3 kg initial body weight, obtained from Blossin SA, 13-Aubagne, France) were freely housed in facilities with natural daylight and free access to water for a two-week acclimatizing period prior to treatment. All procedures were performed on animals anesthetized with tiletamine (2.5 mg/kg), zolazepam (2.5 mg/kg), and atropine (50 μg) IM, followed by an IV infusion of propofol 0.6 mg/kg/10 ml (μg) or 2 mg/kg/10 ml (baboon) in isotonic Ringer-Lactate solution (2 ml/min). The animals were intubated and ventilated, and anesthesia was maintained throughout the procedures with gaseous sevoflurane (1.2%) and sufentanil (2.5 pg/kg/h). In general, percutaneous access was obtained via Seldinger's approach by puncturing the right superficial femoral artery (SFA) under Doppler ultrasound guidance and introducing a 10 cm long standard 5F vascular sheath (Radiofocus Terumo Corporation, Tokyo, Japan). Aseptic techniques were used throughout the procedures. Antibiotics (amoxicillin:clavulanic acid, 1 g:200 mg, in 20 ml, IV) were administered both before and after the procedures.

Radiofrequency ablation (RFA) studies were carried out on one or more kidney areas of the animal models. The studies included the following steps, though not every experiment included every step:

(1) Pre-RFA imaging of the animals;

(2) Embolization of specified kidney areas;

(3) Post-embolization imaging to assess the realized embolization (this step was performed only in experiments in which embolization was performed);

(4) Radiofrequency ablation (RFA) of specified kidney areas;

(5) Post-RFA imaging;

(6) Euthanasia of the animals and surgical ablation of the kidneys; and

(7) Electron microscopy and histology of harvested kidneys.

Pre-RFA imaging was performed as follows. Anesthetized animals were placed in a dorsal recumbent position, with their lower limbs folded down onto the table, in the standard radiological posture. Digital subtraction angiography (DSA) agiograms were obtained using a digitized subtraction angiographic stenoscope system (General Electric Medical Systems, Minneapolis, Mn.). A 5F (65 cm) angiographic UF catheter (Cordis, Miami, Fla.) was advanced into the abdominal aorta over a 0.035 inches hydrophilic guide wire (Radiofocus Terumo Corporation, Tokyo, Japan), and an aorto-renal angiogram was obtained by injecting 25 ml Hexabrix (Guerbet Inc.) at 12 ml/sec (pigs) or 10 ml Hexabrix at 10 ml/sec (baboons). Animals were also given a CT-scan with spontaneous contrast using a helix CT-scanner Tomoscan M (Philips, The Netherlands).

Embolization was performed as follows. An interlobar artery of the left or right kidney was catheterized according to standard radiology procedure and then embolized with Marsembol or the composition of Example 3 via a Rapid Transit microcatheter (Cordis, Miami, Fla.). The quantity of the composition required to obtain complete embolization was 1.7±0.2 ml. The microcatheter was flushed with saline after injection of the composition.

Radiofrequency ablation was performed as follows. The animals were given general anesthesia, intubated, ventilated, and then placed in a ventral position with four return electrodes connected to the RF generator of the RF ablation system (RF 3000, Boston Scientific). A peripheral venous line was placed to infuse fluids, drugs, and the contrast medium. The position of the needle electrodes (LeVeen Superslim, 2 cm array, Boston Scientific) and their deployment in the renal cortex were assessed using ultrasound and CT-scan. The RF ablation thermal protocol increased power in 10 watt increments every minute starting from 20 watts until the first roll-off was observed. Then, starting at half the power of the first roll-off, power was increased in the same increments until the second roll-off was observed. Roll-offs for some experiments using no embolization, embolization with Marsembol only, and embolization with the composition of Example 3 only are provided in Table 1.

TABLE 1

| | Baboon | Pig |
|---|---|---|
| No Embolization | Roll-off 1: 5.5 min/50 W | Roll-off 1: 3.5 min/50 W |
| | Roll-off 2: 4.1 min/45 W | Roll-off 2: 2.5 min/40 W |
| Marsembol | Roll-off 1: 4.5 min/50 W | Roll-off 1: 3.4 min/50 W |
| | Roll-off 2: 3.9 min/40 W | Roll-off 2: 2.1 min/40 W |
| Example 3 | Roll-off 1: 4.1 min/50 W | Roll-off 1: 2.1 min/50 W |
| | Roll-off 2: 3.1 min/40 W | Roll-off 2: 1.5 min/35 W |
| Kill Zone Volume | 35.15 +/− 11.28 | 27.28 +/− 9.14 |
| (% kidney volume) | | |

In the swine (N=6), RF treatment procedure were applied to the inferior pole of the left and right kidneys (previously embolized or not) as follows: Control (no embolisation) versus mrs-embolisation (N=2), control versus mrs-MWCNT-embolisation (N=2) and mrs-embolisation versus mrs-MWCNT-embolisation (N=2). The same 6-sites protocol was applied to 3 baboons, but in both the superior and inferior lobes in each kidney of each animals.

Post-RFA imaging was performed as follows. Digital subtraction angiography of specified kidney areas was carried out as described above. In some experiments, two surveillance CT-scans were also carried out, one immediately after the procedure (DO) and the other after four weeks (D28), with and without injection at the nephrographic stage of the contrast medium at the level of the kidneys as detected on the radiologic screens (1.5 ml/kg per injection) to study the shape, size, and enhancement of the ablation zone and to assess whether there were local or regional complications. Renal parenchymal atrophy was investigated using a flat panel, three-dimension rotational study with multiplanar and volume rendering reconstructions (Innova 3100 imaging, General Electric Medical Systems, Minneapolis, Mn.).

In the aorta above the renal arteries, the contrast medium was delivered from a pig tail catheter (90 ml at 4 ml/sec) under helix CT acquisition (axial 2.5 mm slices for 180 seconds)

Euthanasia and kidney removal were typically carried out as follows. After RFA treatment, the animals were given about four hours to rest to allow time for the activation of caspases such as Caspase 3. Euthanasia was then carried out with an intravenous bolus injection of 15 mg of midazolam and 25 mg of chlorpromazine with 20 ml of KCl at 15%. The embolized renal areas were located under radiological guidance. Median laparotomy was then performed, and the kidneys were surgically removed and immersed in buffered 10% formalin liquid fixation medium for two days. Kidneys were harvested in an identical manner for electron scanning microscope examination and conventional histology.

Histology was carried out as follows. Under radiological guidance and macroscopic examinations, serially cut 5 mm sagital slices were obtained, encompassing both the embolized zone and the apparently unscathed vicinal kidney. Secondary slices (6-7mm in thickness) were cut at the center, periphery and outside of the ablation zone at 25, 50 and 100% distance relative to the ablation zone. The slides were then examined by a specialized renal pathologist using hematoxylin eosin-safron staining (HES) and two immuno-histochemical markers, anti-CD10 (marker of the microvilli of the proximal convoluted tubuli) and Purified Rabbit Anti-Active Caspase-3 (an anticaspase apoptosis marker).

Fragments for TEM investigations were refixed in glutaraldehyde (2.5%,v/v) in 0.1M cacodylate (ph7.4) buffer for lhr, postfixed in OsO4 2.4% prior progressive alcohol dehydratation. Semi-thin (1 μm) and ultra-thin sections in epoxyresin Epon812 were cut with diamond knifes (Leica UltracutE) and stained with uranyl acetate (5% in water) and lead citrate (M in NaOH,1M). TEM investigations were carried out on a Jeol JEM 1400 microscope (80 kvolts) and image recordings with MegaView3 system (Olympus). Pathologic and IHC examinations were performed on serially cut 4 μm thin sections in paraffin (56° C.Histowax, Goteborg, Sweden) stained with hematoxyllin-eosin-saffranin (HES) and Masson's trichrome. The Histostain®kit system (streptavidin-alkaline phosphatase, DABchromogen) was used to detect active caspase-3 activity (Zymed Laboratories Inc., SanFrancisco, USA). Morphometric analysis was performed using an automatic videoanalyzer Nikon Elipse H600L (21-23).

Data treatment and statistical analyses (Kruskall-Wallis non-parametric anova and Mann-Whitney Utest) were performed by using Systat12 (SPSS Inc, Chicago, Ill.). A non-parametric kernel density estimator was preferred to rule out a functional form on the distribution function curves. Results are expressed as mean+SD (23).

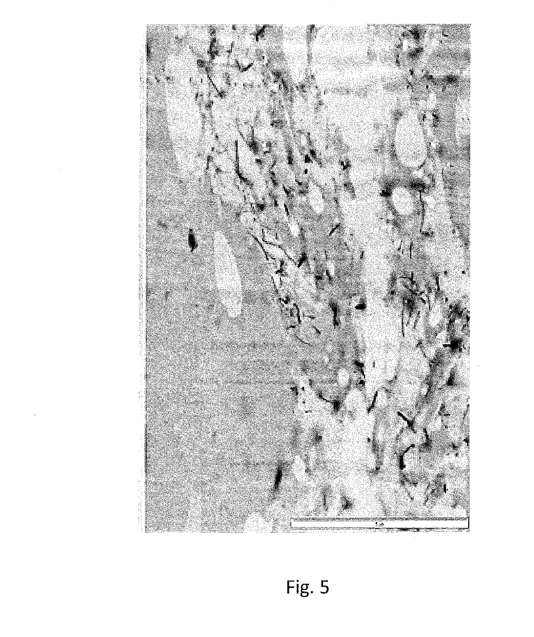

Some effects of RF treatment on the composition of Example 3 are shown in FIG. 5. FIG. 5 displays areas of carbonization of the Marsembol matrix, with increased carbonization near MWCNT-containing sites.

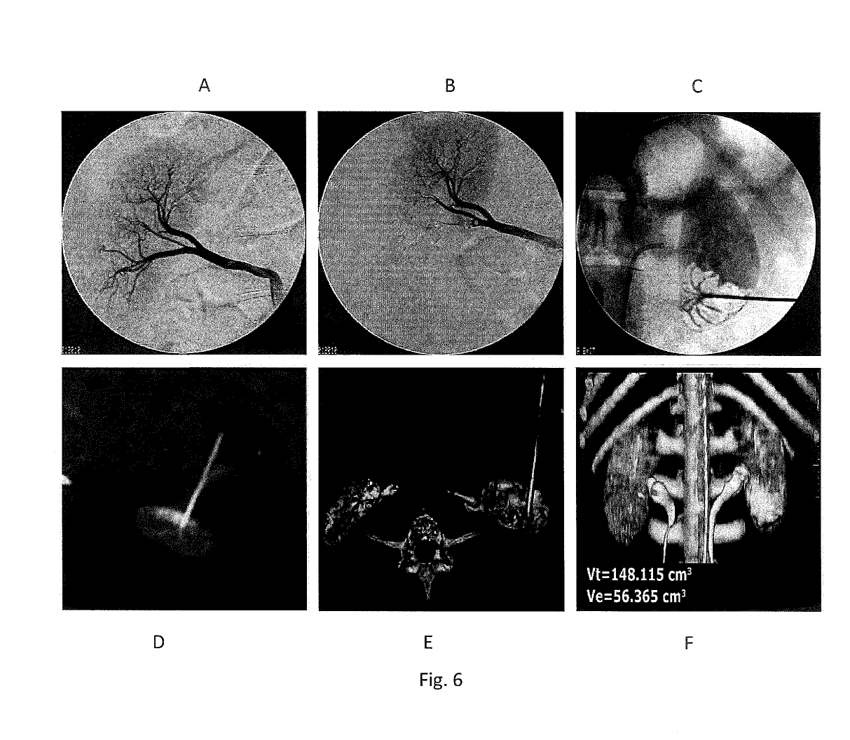

Some results of the RFA studies are shown in FIG. 6. FIG. 6A shows a DSA angiogram of the right renal artery before embolization. FIG. 6B shows a DSA angiogram of the same artery after embolization with Marsembol. The comparison of FIGS. 6A and 6B indicates that the inferior pole of the right kidney was no longer perfused after embolization and was instead occluded by the embolic agent. FIG. 6C shows a DSA angiogram of the left kidney indicating that the lower pole of the kidney was not perfused by the time of perfusing with contrast media. For RFA, the RF needle was placed under both echographic and radiologic guidance exactly in the embolized areas. FIG. 6D is a CT-scan image of the RF LaVeen needle. FIG. 6E shows a three dimensional reconstruction of the embolized kidneys with the RF needle in the right kidney. FIG. 6F shows a three dimensional reconstruction and volumetric analysis of the embolized kidneys, where Vt=volume total and Ve=volume embolized for the right kidney. The results for the left kidney (not shown) were similar.

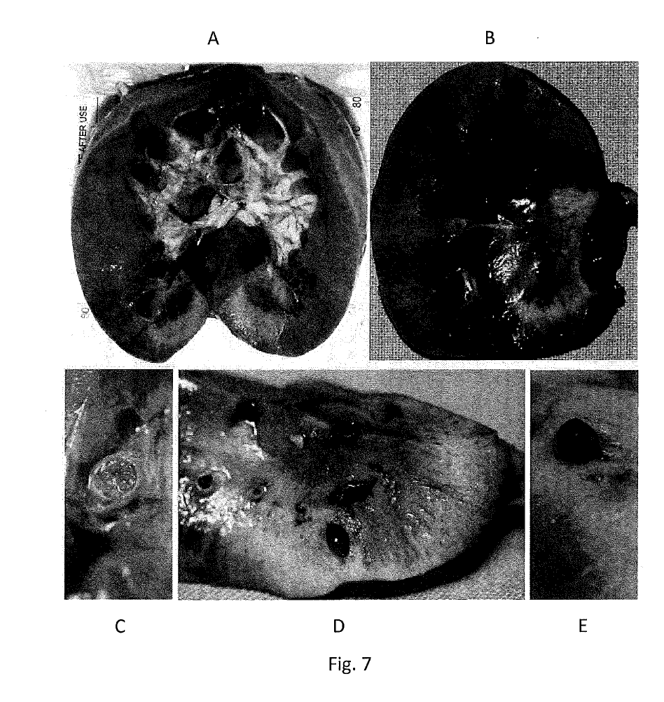

FIG. 7 shows the macroscopic appearance of cross sections of kidneys after RFA with and without embolization using a composite composition prepared in accordance with Example 3. FIG. 7A shows a non-embolized porcine kidney cut open longitudinally. A bright, light brown print of the RFA on the lower pole of the kidney was observed. FIG. 7B shows a non-embolized baboon kidney cut open longitudinally. The cortex thickness in the porcine kidneys was thinner than in the baboon kidneys, in which the nephronic structures occupied most of the kidneys. Also, the baboon kidney exhibited arciform and radial branches of the renal artery through the kidneys, which was not observed in the porcine kidneys. FIG. 7D shows a two-dimensional, orthogonal cut in a baboon kidney treated with RF in the presence of the composite composition of Example 3. The occluded vessel at the frontier of the RF-treated area was light brown, as compared with the black color of the occluded vessel in the RF-treated area itself, as shown in FIG. 7C (occluded vessel outside the RF-treated zone) and 7E (occluded vessel inside the RF-treated zone). FIGS. 7C-E thus indicate that the embolic agent was carbonized (decomposed) in the specific RF-treated zone but was not carbonized outside of the treated zone. FIG. 7E further indicates that the RF treatment led to burning out of the specific treatment zone.

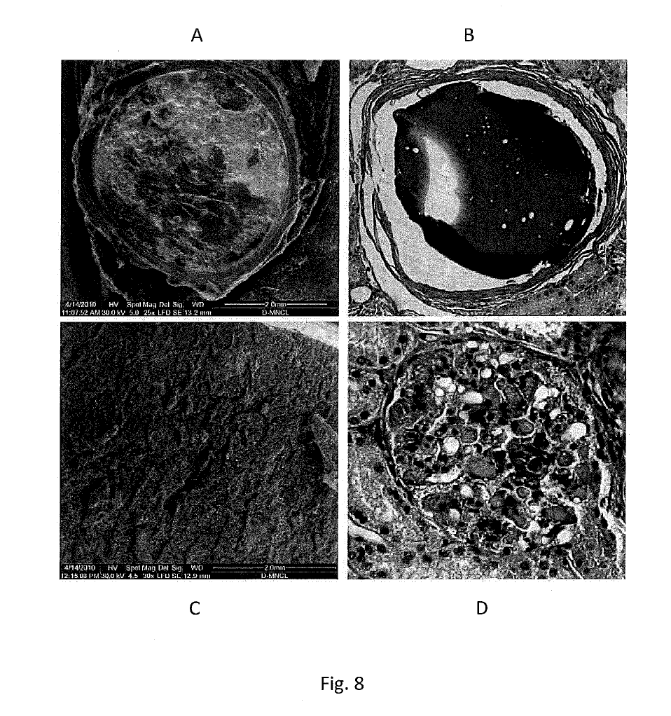

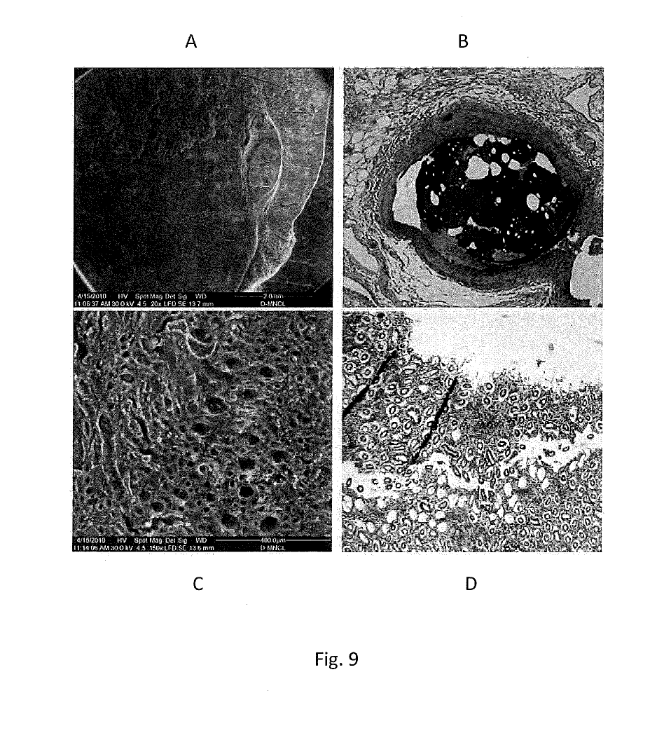

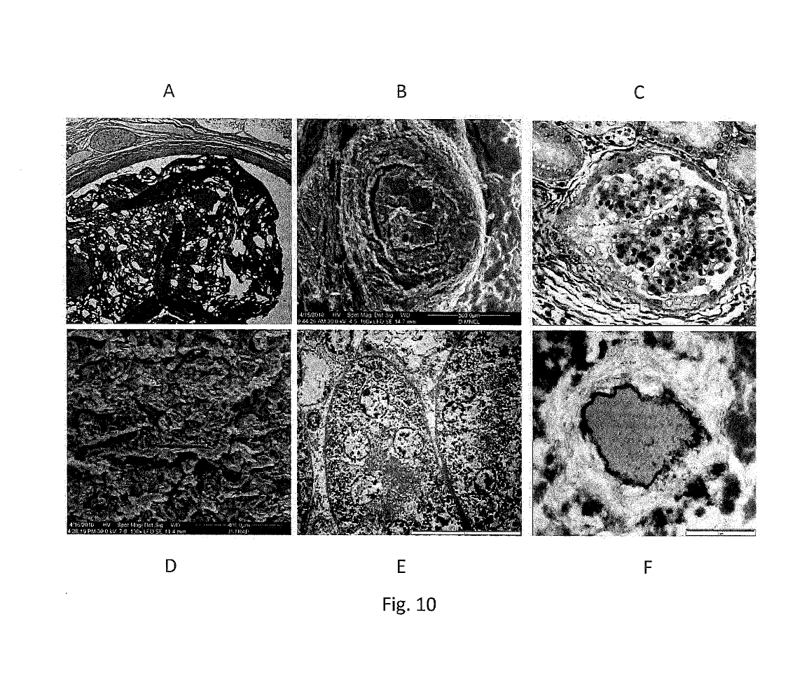

FIGS. 8 through 10 display SEM and conventional histological analyses of harvested kidneys subjected to different treatments. FIG. 8 corresponds to treatments including embolization with Marsembol and the composite composition of Example 3, but not including RFA. FIG. 9 corresponds to embolization with Marsembol followed by RFA. FIG. 10 corresponds to embolization with the composition of Example 3 followed by RFA.

FIGS. 8A and 8C are SEM images, and FIGS. 8B and 8D are conventional histology images. FIGS. 8A and 8B illustrate the amorphous nature of Marsembol in a kidney blood vessel. FIG. 8C illustrates the preservation of all the tubes and nephrons in a control kidney. FIG. 8D illustrates the histological appearance of a nephron containing the composition of Example 3 in the very distal afferent renal artery.

FIGS. 9A and 9C are SEM images, and FIGS. 9B and 9D are conventional histology images. FIG. 9A illustrates the frontier between the RF ablation zone and the immediately neighboring renal tissue. Major destruction of the tissue in the RF-treated zone was observed. FIG. 9B illustrates the histological appearance of a cross-section of a vessel containing the embolic agent plug in the RF-treated zone. The plug was vacuolized at least in some places, indicating that the RF was able to partially destroy the embolic agent. FIG. 9C shows the destroying effect of RF on the tubular structures of the nephron. FIG. 9D shows HES staining at the border of the RF needle. The white space in the image corresponds to burned renal tissue (ashes were lost during tissue processing procedures). Also, the tubular structures were observed to be dissociated from their basal blades.

FIGS. 10B and 10D are SEM images, FIGS. 10E and 10F are TEM images, and FIGS. 10A and 10C are conventional histology images. FIG. 10A illustrates the histological appearance of a cross-section of a vessel containing the embolic agent plug in the RF-treated zone. The highly vacuolized plug indicated that the plug was much more destroyed due to the presence of the MWNTs in the Marsembol embolic agent. FIG. 10B indicates that the vessel wall in the image responded to the treatment by increasing its thickness. It can also be observed that the embolic agent was highly heterogeneous. FIG. 10C illustrates the histological appearance of semi-thin sections (0.5 μm) of a nephronic structure after RF treatment. It can be seen that the embolic agent (dark blue) no longer completely occluded the very distal arterioles. Also, in the vicinal tubes the plasma cell membranes were ruptured but the nuclei membrane remained detectable. FIG. 10D illustrates that the nephronic and tubular structures were preserved in their shape. FIG. 10D displays the appearance of two contiguous tubules, illustrating that cell membranes were destroyed whereas the nuclei membranes were preserved. Both cellular and nuclear contents and organelles were disaggregated. In the upper and left hand side of the central tubules, two small dots in the homogeneous grey color were observed. FIG. 10E illustrates one of these dots, which corresponds to the residual vascular content of the embolic agent. This residual dot was surrounded by structures which aggregated and clustered and had the size and the appearance of MWNTs, suggesting that once the Marsembol embolic agent disaggregated or decomposed, clusters of MWNTs formed. FIG. 10 illustrates that the organelle structures of renal tubules were preserved (external and nuclear membranes were intact), but the subcellular structures were destroyed.

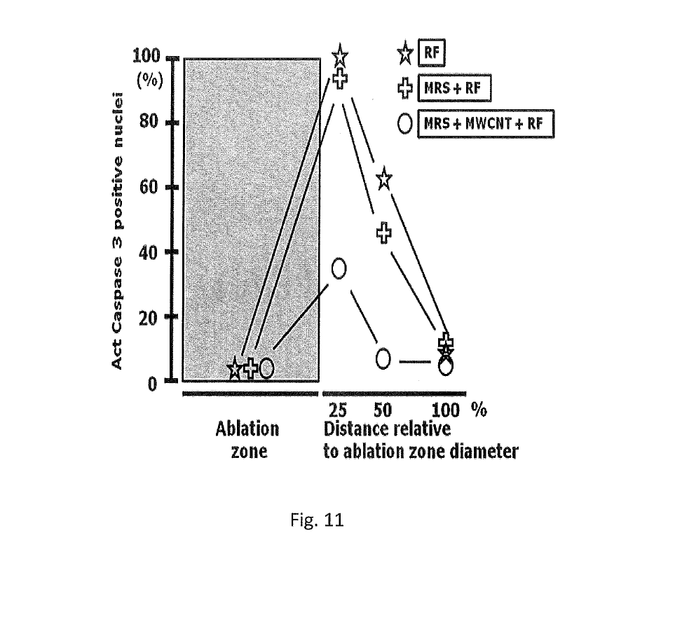

FIG. 11 shows the early (2 h) activated Caspase-3 distribution in kidney tissue after several different treatments, as a function of radial distance from the ablation zone (in units of percentage of the ablation zone diameter): the control experiment (i.e., RF treatment with no embolization, top line marked with stars), embolization with Marsembol followed by RF treatment (middle line marked with crosses), and embolization with the composite composition of Example 3 followed by RF treatment (bottom line marked with circles). The percent of activated Caspase 3 positive nuclei is plotted on the y-axis (positively-stained nuclei compared to the overall number of cells). The x-axis corresponds to the radial distance of the nuclei from the ablation zone. In the ablation zone, virtually all of the cells were killed, regardless of treatment. However, FIG. 11 illustrates that in the immediate surroundings of the ablation zone the level of apoptosis varies based on treatment type. For the control treatment, almost one hundred percent of the cells were turned to apoptosis outside the ablation zone within a distance of about 25% of the diameter of the ablation zone, with a decrease in apoptosis level to normal beyond the apoptotic crown surrounding the ablation zone. In contrast, treatment with the composite composition of Example 3 corresponded to a drastic reduction in apoptosis induction in surrounding tissue.

Various embodiments of the invention have been described in fulfillment of the various objects of the invention. It should be recognized that these embodiments are merely illustrative of the principles of the present invention. Numerous modifications and adaptations thereof will be readily apparent to those skilled in the art without departing from the spirit and scope of the invention.

That which is claimed is:

1. A composite composition comprising:
an embolic agent; and
a plurality of nanoparticles dispersed in the embolic agent, wherein a portion of the nanoparticles are individually dispersed in the embolic agent.

2. The composite composition of claim 1, wherein the nanoparticles are dispersed throughout the embolic agent.

3. The composite composition of claim 2, wherein the nanoparticles are dispersed substantially uniformly throughout the embolic agent.

4. The composite of claim 1, wherein the nanoparticles have an aspect ratio of 10 to 1,000.

5. The composite composition of claim 1, wherein the nanoparticles have a length ranging from 500 nm to 1.5 mm.

6. The composite composition of claim 1, wherein the nanoparticles comprise carbon nanotubes.

7. The composite composition of claim 1, wherein the nanoparticles are present in the embolic agent in an amount ranging from 0.1 μg/ml to 5 mg/ml.

8. The composite composition of claim 1, wherein the embolic agent has a kinematic viscosity permitting intravascular introduction of the composite composition through a microcatheter having an inner diameter ranging from 100 μm to 1500 μm.

9. The composite composition of claim 1, wherein the embolic agent comprises collagen, thrombin, lipiodol, a gelatin or alginic acid or combinations thereof.

10. A treatment system for diseased tissue comprising:
a source of radiofrequency energy; and
a thermal induction agent operable for positioning in the diseased tissue, the thermal induction agent comprising a material having a dielectric loss factor greater than a dielectric loss factor of the diseased tissue.

11. The treatment system of claim 10, wherein the material of the thermal induction agent comprises a composite composition comprising an embolic agent and a plurality of nanoparticles dispersed in the embolic agent, wherein a portion of the nanoparticles are individually dispersed in the embolic agent.

12. The treatment system of claim 11, wherein the nanoparticles are dispersed throughout the embolic agent.

13. The treatment system of claim 12, wherein the nanoparticles are dispersed substantially uniformly throughout the embolic agent.

14. The treatment system of claim 11, wherein the nanoparticles have an aspect ratio of 10 to 1,000.

15. The treatment system of claim 11, wherein the nanoparticles have a length ranging from 500 nm to 1.5 mm.

16. The treatment system of claim 11, wherein the nanoparticles comprise carbon nanotubes.

17. The treatment system of claim 11, wherein the nanoparticles are present in the embolic agent in an amount ranging from 0.1 μg/ml to 5 mg/ml.

18. The treatment system of claim 10, wherein the source of radiofrequency energy is a radiofrequency probe.

19. A method of treating diseased tissue comprising:
disposing in the diseased tissue a material having a dielectric loss factor greater than a dielectric loss factor of the diseased tissue to define a predetermined cellular killing zone in the diseased tissue; and
providing thermal energy to the diseased tissue by exposing the diseased tissue and the material to radiofrequency energy.

20. The method of claim 19, wherein the predetermined cellular killing zone is restricted to the region of the material of greater dielectric loss factor.

21. The method of claim 19, wherein the predetermined cellular killing zone includes the region of the material of greater dielectric loss factor and an adjacent region in the tissue having dimensions up to about 50% the diameter of the region of greater dielectric loss factor material.

22. The method of claim 19, wherein the material of greater dielectric loss factor comprises a composite composition comprising an embolic agent and a plurality of nanoparticles dispersed in the embolic agent, wherein a portion of the nanoparticles are individually dispersed in the embolic agent.

23. The method of claim 19 further comprising ablating at least a portion of cells in the predetermined cellular killing zone.

24. The method of claim 19, wherein the radiofrequency energy is supplied by a radiofrequency probe.

25. A method comprising:
reducing damage to non-diseased tissue adjacent to diseased tissue during radiofrequency thermal ablation of the diseased tissue in an ablation zone by restricting formation of an apoptotic cellular region in the non-diseased tissue resulting from the radiofrequency thermal ablation, wherein restricting comprises:
disposing in the diseased tissue prior to application of radiofrequency energy a material having a dielectric loss factor greater than dielectric loss factor of the diseased tissue.

26. The method of claim 25, wherein the material is present in the diseased tissue in an amount sufficient to thermally ablate at least a portion of the diseased tissue.
